(19) United States
(12) Patent Application Publication
(10) Pub. No.: US 2025/0246297 A1
(43) Pub. Date: Jul. 31, 2025

(54) MEDICAL SYSTEM AND METHOD FOR CONTROLLING A SYSTEM COMPRISING AT LEAST TWO MEDICAL DEVICES, HAVING IMPROVED MODULARITY DUE TO NEAR-FIELD COMMUNICATION

(71) Applicant: KARL STORZ SE & Co. KG, Tuttlingen (DE)

(72) Inventors: Andres ASPER, Tuttlingen (DE); Andreas BAYER, Tuttlingen (DE); Harry RUTSCHMANN, Tuttlingen (DE)

(73) Assignee: KARL STORZ SE & Co. KG, Tuttlingen (DE)

(21) Appl. No.: 18/866,895

(22) PCT Filed: May 8, 2023
(86) PCT No.: PCT/EP2023/062155

§ 371 Date: Nov. 18, 2024

(30) Foreign Application Priority Data
May 19, 2022 (DE) 10 2022 112 646.3

(51) Int. Cl. G16H 40/60; H04W 4/80

(52) U.S. Cl. CPC G16H 40/60; H04W 4/80

(57) ABSTRACT

The invention relates to a system comprising at least two medical devices and to a method for controlling a system comprising at least two medical devices, the at least two medical devices each comprising at least one near-field wireless communication interface, which near-field wireless communication interfaces are arranged in or on the medical devices in such a way that, when the medical devices are arranged in at least one particular arrangement relative to one another, a near-field wireless communication interface of one device is arranged within the communication range of a near-field wireless communication interface of another device.

### CROSS-REFERENCE TO RELATED APPLICATIONS

This application is the U.S. national stage of PCT/EP2023/062155 filed on May 8, 2023, which claims priority of German Patent Application No. DE 10 2022 112 646.3 filed on May 19, 2022, the contents of which are incorporated herein.

### TECHNICAL FIELD

The disclosure relates to a method for controlling a system with at least two medical devices, wherein the at least two medical devices each have at least one near-field wireless communication interface. Furthermore, the disclosure relates to a system which is controlled by this method.

### BACKGROUND

Within the field of medical devices, devices with the same or similar basic functions or basic functionalities at different levels of complexity or with a different range of functions were previously regularly developed and marketed as independent but different devices. This led to the situation that simple medical devices could be used to perform correspondingly simple tasks and functions, whereas more complex processes could be carried out or supported using devices that were correspondingly further developed or provided with additional functions and functionalities. However, this led to the situation where medical facilities, such as doctor's offices, hospitals, or care facilities, had to purchase, maintain, and service both devices with a simple design or low level of integration and complex and sophisticated devices with a high level of integration, even though the basic functions or basic functionalities of the different devices are similar or even identical. However, this results in high investment costs and large amounts of maintenance. Furthermore, medical personnel must be able to operate all devices safely and reliably.

It is also already known from the prior art to create a data coupling or connection between medical devices, wherein different devices fulfill different static or fixed roles or functions. Such a system is known, for example, from U.S. Pat. No. 10,292,661 B1. A coupling or data connection of different, identically designed modules to a carrier or receiving structure in the form of a connection of a plurality of infusion pumps with a type of docking station is already known from EP 3 010 193 A1.

Within the field of respiratory gas humidification devices, WO 2015/167347 A1 is also known, in which different modules can be switched on or off on the basis of measured values.

However, the approaches known from the prior art have the disadvantage that, despite a data connection or communication between the individual devices or individual modules, the range of functions or the scope of integration of the functionalities is not influenced, in particular expanded, by the data connection or coupling.

### SUMMARY

Against this background, the object of the present disclosure is to provide a method for controlling a system comprising at least two medical devices and a medical system having at least two medical devices, which overcome the disadvantages of the prior art and in particular allow for modular functional integration or an increase in the range of functions.

This object is achieved by the features of independent claims 1 and 18. De-velopments of the disclosure form the subject matter of the dependent claims, the following description, and the description of the figures.

With regard to the method for controlling a system comprising at least two medical devices, the object is achieved in that the at least two medical devices each have at least one near-field wireless communication interface, which is arranged in or on the medical devices such that, in at least one specific arrangement of the medical devices relative to one another, a near-field wireless communication interface of one device is arranged within the communication range of a near-field wireless communication interface of another device, and vice versa, wherein the method comprises the following method steps:

In a first method step, the medical devices are operated in a first independent operating mode in the form of an individual operating mode, in which each device performs or provides an individual function as long as the near-field wireless communication interface does not detect a connection or possible connection to a further near-field wireless communication interface.

In a further method step, at least two devices are identified in a specific arrangement relative to one another by means of the near-field wireless communication interfaces.

In a further method step, the at least two medical devices are then operated in a second operating mode in the form of a coupled operating mode, wherein the coupled operating mode of at least two devices performs or provides a combined function that cannot be performed or provided by the individual devices operated in the coupled operating mode if and/or as long as at least one near-field wireless communication interface has established a connection to a further near-field wireless communication interface or detects a possible connection to a further near-field wireless communication interface.

The method according to the disclosure therefore allows different medical devices to be operated both as medical devices with rather basic or simple functionality, specifically as a device in an individual operating mode, but at the same time, in the case of a connection or possible connection with another, correspondingly set up and designed medical device, to be operated as a combined medical device with more complex functionality, wherein this greater complexity or the greater range of functions is provided by operating the medical devices in a second operating mode, specifically in a coupled operating mode.

It should be noted that the actual operation or the actual function of the individual medical devices in the individual operating mode and in the coupled operating mode may only be changed slightly, which is also relatively self-evident due to the limited resources and functionalities of the individual device, wherein it is precisely through the combined or coupled use of the individual devices that the coupled operating mode, or in other words a common coupled operating mode, is created or achieved.

To illustrate the method, an exemplary embodiment or application shall be described below which describes the use of roller pumps in a method according to the disclosure. However, the disclosure shall in no way be limited to this example use or design. Rather, the description serves to illustrate a preferred embodiment.

Therefore, in a method according to the disclosure, two individual roller pumps, which are individually widely used as medical devices, could be used in a first operating mode, viz., individually in the individual operating mode, and independently as individual roller pumps. However, as soon as the two roller pumps are transferred into a specific arrangement relative to one another, which is detected or determined by means of the near-field wireless communication interfaces, it is possible for the two roller pumps to perform the function of a double roller pump, wherein, in order to perform the function of the double roller pump, the two roller pumps or the individual roller pumps are placed or transferred into a coupled operating mode, which then enables not only operation as a single roller pump, but especially joint or coupled operation as a double roller pump if and/or as long as a connection or possible connection of the near-field wireless communication interfaces of the two roller pumps exists or has been established.

For example, in the individual operating mode, the roller pumps could work as individual suction or flushing pumps, as required. In the coupled operating mode, they could then act as a commonly regulated suction and flushing pump, which, for example, regulates the suction and flushing of fluid during a medical procedure in such a way that a pressure in a cavity is not exceeded.

This example also makes it clear that the method steps according to the disclosure can be carried out or executed both in the example and in general over a relatively long period of time or time window and, if appropriate, also interrupted by phases of inactivity of the system and/or the medical devices. In other words, this means that the method steps according to the disclosure do not necessarily have to directly follow one another. Thus, in the example of the roller pumps as medical devices, it may quite possibly, or even in the majority of cases, be advantageously provided that, after use of the medical devices or roller pumps in an individual operating mode, a state of inactivity and/or a state of maintenance or preparation or processing initially take place for one or at least two roller pumps before the at least two medical devices or two roller pumps are transferred to the coupled operating mode and operated therein, in which the identification of the specific relative position of the medical devices to one another is detected by means of the near-field wireless communication interface.

It is also equally apparent that a substantially reversed method sequence is or can be possible and advantageous according to the disclosure, in which-again possibly interrupted by other method steps and accordingly carried out within a larger time window—the medical devices switch to the individual operating mode and the provision of the individual function after the identification of the specific relative arrangement and the implementation of the coupled operating mode caused or effected thereby and the provision of a combined function, if the identification of the specific arrangement of the devices relative to one another is done away with or discontinues.

In principle, different near-field wireless communication points and associated protocols or communication protocols are possible for the method according to the disclosure. However, it is particularly desirable that the corresponding range of the near-field wireless communication interfaces be reduced either fundamentally and for technical reasons or by appropriate technical influence—for example, deliberate shielding or attenuation—to such an extent that the range of the near-field wireless communication interface, depending upon the arrangement and orientation in or on the medical device, does not exceed a typical, possibly even standardized, device dimension in one spatial direction and is preferably limited to a maximum of three-quarters the size of the device dimension in one spatial direction, particularly preferably to less than half of the device dimension in one spatial direction.

As a result, in a particularly advantageous manner, the clear detection of the at least two medical devices in a specific relative arrangement can be detected or identified when, and preferably only when, the medical devices are located or arranged in close spatial proximity, in particular in an arrangement where they are immediately adjacent to one another. This can prevent ambiguities in which a medical device or the corresponding near-field wireless communication interface identifies a plurality of connection possibilities or connection possibilities to other near-field wireless communication interfaces, and thus the system and the method are under-determined or insuffi-ciently defined, since in this case it would not be possible or not readily possible to detect which medical devices are to be transferred to the coupled operating mode with one another and are to provide the combined function.

Technologies that originally have a correspondingly short range are based, for example, upon “Near-Field Communication” (NFC) technology. But even quite ru-dimentary near-field wireless communications, e.g., in the form of a magnetic contact to form a reed relay, inherently have these correspondingly limited ranges and are to also be expressly understood as near-field wireless communication interfaces in the sense of the present disclosure.

Other technological implementations, such as wireless communications based upon RFID technology, Bluetooth technology, or optical communication interfaces, would have to be adapted for preferred suitability in the method according to the disclosure such that they have a range, preferably a reduced range, as described above.

According to a first advantageous embodiment of the method, it can be provided that the identification of two devices in a certain relative arrangement be achieved as a result of periodically repeated connection attempts in which at least some of the near-field wireless communication interfaces make an attempt to establish a connection to a further near-field wireless communication interface.

This makes it particularly advantageous to fully automatically or at least par-tially automatically transfer to the second operating mode, specifically the coupled operating mode. This is because regularly querying or verifying connections or connection possibilities to other near-field wireless communication interfaces means that, as soon as at least two medical devices are arranged in the specific arrangement relative to one another, the automatic and/or periodically repeated connection attempts lead to the detection of the connection or connection possibility of the near-field wireless communication interface and thus to the detection of the specific position of the medical devices relative to one another, which then in turn leads to the at least two medical devices being transferred to the second operating mode.

In a further, particularly preferred embodiment, it can be provided that the second operating mode be determined by the number and/or position and/or type of near-field wireless communication interfaces that have established a connection to a further near-field wireless communication interface or detect a connection possibility. The determination of the second operating mode on the basis of the number and/or positions and/or type of each connected or connectable near-field wireless communication interface allows for a plurality of different second operating modes and thus a plurality of different combined functions of individual medical devices, wherein a clear determination and selection of each second operating mode and the associated combined function is achieved by a specific arrangement of the medical devices relative to one another, which is then detected or identified by different positions, numbers, and/or types of near-field wireless communication interfaces, even if the medical devices used would also allow or could allow for one or more alternative combined functions.

For example, a medical device could have two or three near-field wireless communication interfaces on or in a housing, e.g., on the base and/or cover side, wherein a corresponding combinatorial selection of the second operating mode is then made on the basis of the near-field wireless communication interfaces that establish a connection or detect a connection possibility.

In a further, likewise particularly advantageous embodiment of the method, it can be provided that, in the second operating mode, a first device perform a control function for at least one further device and send control data to the further device and/or receive operating data from the further device for this purpose. In other words, this means that, in the second operating mode, one device preferably assumes a master function for performing the coupling function, whereas at least one other device performs a slave function for the coupling function. As a result, coupling functions in particular can be achieved in a particularly advantageous manner in the second operating mode which go beyond the range of functions of the individual device or the individual function in the individual operating mode, since not only is the corresponding relative arrangement of the medical devices detected, but also information, in particular in the form of data, can be exchanged between the devices in the second operating mode and reused accordingly.

According to a further, likewise advantageous embodiment of the method, it can be provided that the control function be assigned dynamically depending upon the location or position of the near-field wireless communication interface that has established a connection to a further near-field wireless communication interface or detects a connection possibility to such an interface. As a result, the arrangement of the medical devices relative to one another and the near-field wireless communication interfaces that are or can be connected to one another as a result can particularly advantageously be crucial for determining which medical device performs the control function and which medical device or which medical devices is/are controlled or influenced by the device having the control function.

Alternatively or additionally, in an advantageous embodiment of the method, it can also be provided that the control function be assigned dynamically depending upon the type of near-field wireless communication interface that establishes or detects a connection or a connection possibility to a further near-field wireless communication interface. This may be particularly, but by no means exclusively, advantageous when different types of near-field wireless communication interfaces are present or installed in a medical device.

Even then, the dynamic assignment of the control function can be determined or specified indirectly via the different arrangement of different near-field wireless communication interfaces through the spatial arrangement or relative arrangement of the medical devices with respect to one another.

According to a particularly preferred embodiment variant, it can further be provided that the control data and/or the operating data be exchanged via the near-field wireless communication interface. In this case, it is accordingly expedient, if not necessary, that not only a connection possibility be detected, but a communication connection actually be established, between the near-field wireless communication interfaces which, together with one another or collectively, cause or effect the operation of each of the medical devices in the second operating mode.

However, in different embodiments, it can still be advantageous if the connection between the near-field wireless communication interfaces is made immediately or immediately after the detection of the at least second interface each time or if, initially, only the connection possibility for switching to the second operating mode is suf-ficient, and the actual connection is only established when operating data and/or control data are to be exchanged. Accordingly, it can also be provided that the actual coupling or the actual establishment of a communication connection between the interfaces only take place temporarily or that the connection be interrupted several times without the second operating mode being terminated, as long as the connection possibility is still detected or identified after the connection has been terminated. In the latter embodiment, the connection can be particularly advantageously established and maintained only if and as long as control data and/or operating data need to be exchanged.

In a likewise particularly advantageous development, it can also be provided that those control data and/or those operating data which, if possible, must be available as real-time data for the second operating mode of the medical devices be selectively exchanged via the near-field wireless communication interfaces. This enables a particularly effective exchange of real-time data, since said near-field wireless communication interfaces are, unlike other communication channels and communication technologies, particularly suitable for transmitting relatively small amounts of data particularly quickly.

In a further preferred embodiment of the method, it can be provided that, when operating at least three medical devices in a second operating mode, at least one medical device comprise two near-field wireless communication interfaces and cause control and/or operating data to be passed between at least two other devices. As already mentioned above, it can be particularly advantageous for the present method if each of the medical devices are moved to the second operating mode due to their immediate spatial proximity or adjacency, and, at the same time, a coupling mode, which is possibly dependent upon the position or upon the relative arrangement, and a corresponding coupling function are performed or activated. However, this also means that, as a rule, there are no highly branched networks of medical devices or even networks in which a data connection is potentially provided between every medical device and every other medical device, but that, often or usually, especially with more than two medical devices, a series connection or series connectivity is achieved or attained via the near-field wireless communication interfaces. Even if, rather excep-tionally, a cross-shaped or star-shaped structure or a corresponding connection network having medical devices should be created, for example, the present embodiment can particularly effectively enable the transmission of control and/or operating data by central medical devices or medical devices that have or can have several connections and their corresponding near-field wireless communication interfaces forwarding said data to medical devices arranged further out or more remotely. For example, the devices can collect the data via a near-field wireless communication interface on one side of the device and forward it via a further near-field wireless communication interface on another side of the device.

In a further, particularly advantageous embodiment of the method, it can be provided that control data and/or operating data be exchanged via an additional, preferably wired, communication interface, preferably a bus system, in particular Ethernet protocol-based communication. Depending upon the design of the system or each medical device and also depending upon the safety relevance of the respective control and/or operating data, it can be provided that the respective data be exchanged and processed only via the additional communication interface so as to be duplicated via the near-field wireless communication interface and via the additional communication interface, or only via the near-field wireless communication interface.

A further embodiment of the method particularly advantageously provides that, when changing to or activating the second operating mode, a verification of the connection or connection possibility of the near-field wireless communication interfaces be carried out via an additional, preferably wired, communication interface. For many medical devices, it is already possible to operate them in a network or to connect them to a network via wired interfaces. For example, bus systems or Ethernet protocols and associated interfaces are already known for connecting the medical devices to one another. These or similarly designed communication interfaces and connections established thereby can be used in a particularly advantageous manner to provide additional verification in addition to the detection of a relative arrangement of the medical devices by means of the near-field wireless communication interfaces, before transfer of the medical devices to the second operating mode is initiated, carried out, or completed. This significantly improves the safety of the system and, in particular, prevents or prohibits the unintentional transfer of the medical devices to the second operating mode. In other words, a verification, e.g., as a so-called “handshake,” can be performed via the additional communication interface to confirm the specific spatial arrangement detected by the near-field wireless communication interfaces. Conversely, the “handshake” could also take place via the near-field wireless communication interface, and only then could further communication take place or be enabled via the additional communication interface.

A further, particularly advantageous embodiment of the method can provide that, when changing to or activating the second operating mode, a verification be carried out by detecting attachments or consumables, specific to the coupled operating mode, of the devices, wherein the specific attachments or consumables of the devices are in turn particularly preferably detected or detectable by the device via corresponding mechanisms. In the example already mentioned above of two roller pumps which are combined in the second operating mode to form a double roller pump or which perform the coupling function of a double roller pump, it can be provided, for example, that coded cassettes be used as consumables for each roller pump, wherein the coded cassettes can be detected or identified by the device itself. Accordingly, in the present embodiment, a corresponding detection of the coded cassettes as consumables or similar consumables or attachments may represent a prerequisite or a further verification stage when changing or intending to change to the second operating mode. Conversely, switching to a second operating mode may require the use of different consumables, such as a different hose set. This can be displayed to the user after the second operating mode has been activated. The coupling function can be performed when the correct consumable has been attached and detected by the devices.

According to a further, particularly advantageous embodiment of the disclosure, it can be provided that, when switching to or activating the second operating mode, a user input must be made on at least one device, preferably a device that performs or fulfills the control function in the second operating mode, to confirm the change in operating mode, and preferably the at least one device issue a request for a user input via an output unit for this purpose. This also further increases operational safety and effectively prevents accidental, unintentional, or premature switching to the second operating mode. An optical output unit, such as a display, can be used as the output unit. Combined output and input units, e.g., in the form of touch displays, can also be used for both the request and the user input.

In a further, likewise preferred embodiment of the method, it can be provided that, when changing to or activating the second operating mode, an output, preferably a display, on an output unit of at least one device be made which outputs or reveals to a user that operation is taking place in the second operating mode. The output for indicating that the at least two medical devices are operating in the second operating mode, i.e., in the coupled operating mode, can preferably be carried out when the pre-vious verification stage(s) for detection purposes and for switching to the second operating mode is/are complete or has/have been fully executed. Accordingly, the output according to the present embodiment can particularly preferably also be carried out after a user input according to the embodiment described above, although a user input is not a mandatory requirement. An optical output unit, in particular a display, can preferably be used as the output unit. However, other output units and therefore other types of output can also be used. For example, an acoustic output unit can generate and output an acoustic output.

In a further, particularly preferred embodiment of the method, it can be provided that, depending upon the operating mode of a medical device, an output unit and/or an input unit, preferably a combined input and output unit, be adapted to the operating mode, in particular automatically, in order to enable the output and/or input of settings and operating data in a manner adapted to each of the operating modes. This particularly facilitates the operation and monitoring of the medical device or medical devices, since the user does not have to ensure that the output unit and/or input unit is adapted to each operating mode, but, rather, this adaptation is made automatically. In order to enable adaptation of the output unit and/or input unit, programmable output and/or input units can preferably be used. For example, programmable display units and/or input units with programmable or variable input functions can be used.

In a further, likewise preferred embodiment of the method, according to the example described at the outset, it can be provided that the first and second medical devices comprise a pump, preferably a roller pump, and, in the first operating mode, a pump functionality, preferably a suction or flushing pump functionality, be provided, wherein, in the second operating mode, a suction-flushing-pump functionality of the two roller pumps is provided in a coupled operating mode, preferably as a double roller pump.

As already shown above, this embodiment has the advantage that the user does not have to purchase, maintain, or train personnel with regard to special double roller pumps, since only individual roller pumps have to be purchased and maintained, which can then, if required and after being appropriately transferred to the coupled operating mode, provide the combined or joint functionality of a double roller pump as a suction-flushing pump.

In a further, particularly desirable variant of the method, it can be provided that a third medical device comprise a hose valve and provide a valve functionality in the first and second operating modes. In this case, it can, however, particularly advantageously be provided that, in the second operating mode, the valve functionality be controlled or influenced by a correspondingly adjacent medical device with a control function, such as that described above, such that the hose valve is also able to con-tribute to or play a part in a combined function of the medical devices in the second operating mode.

The object mentioned at the outset is achieved with respect to a medical system in that the medical system comprises at least two medical devices, each of which has at least one near-field wireless communication interface, a control apparatus, and a data memory, which are configured to execute computer programs to carry out a method according to any one of the preceding embodiments.

Particularly preferably, the control apparatus is designed as a central control apparatus (CPU).

### BRIEF DESCRIPTION OF DRAWINGS

FIG. 1: shows an exemplary embodiment of a medical system according to a first embodiment;

FIG. 2: shows an exemplary flowchart of a method according to the disclosure according to a first embodiment.

### DETAILED DESCRIPTION OF PREFERRED EMBODIMENTS

The disclosure will be explained below by way of example on the basis of only exemplary embodiments, which in turn are shown only schematically.

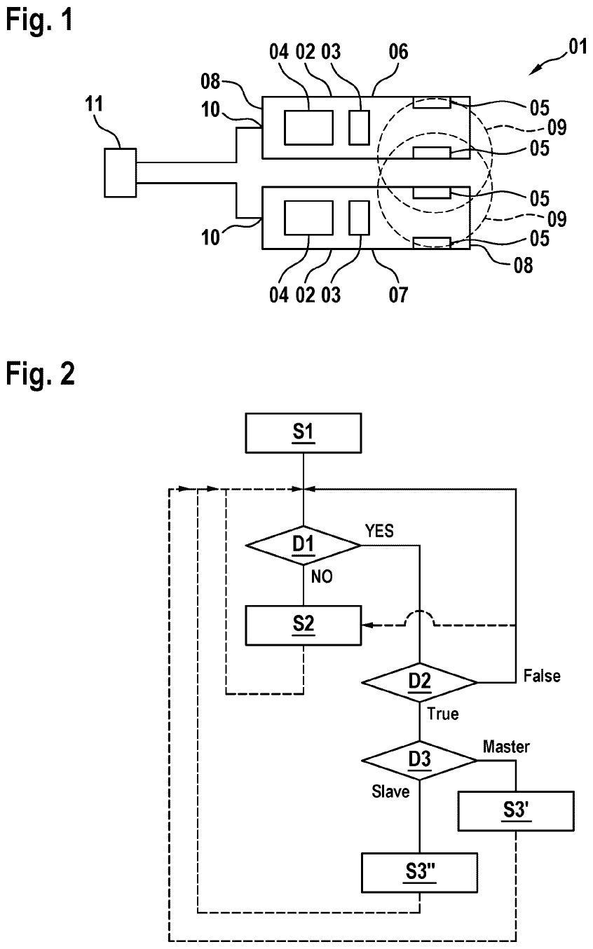

FIG. 1 shows a medical system 01 having two medical devices 02. In the example in FIG. 1, the medical devices 02 are assumed to be designed as roller pumps, which can each individually provide or ensure pump functionality as an individual function in a first operating mode or individual operating mode. The two roller pumps can be operated with removable or exchangeable consumables 03, preferably in the form of coded cassettes. The medical devices 02 also have output units 04 with which settings, parameters, and operating data can be displayed or output.

The two medical devices 02 also have near-field wireless communication interfaces 05, each of which is arranged near a cover 06 or base 07 of a housing 08 inside the device 02. The near-field wireless communication interfaces 05 are arranged such that, when the medical devices 02 are arranged directly on top of one another, as shown in FIG. 1, they are arranged substantially congruently one above the other.

Furthermore, in FIG. 1, the range 09 of the two near-field wireless communication interfaces 05 facing one another is shown in dashed lines. It can be seen that the ranges 09 are implemented in such a way that each near-field wireless communication interface 05 can detect or reaches a further near-field wireless communication interface within the corresponding range only if the medical devices 02 are arranged directly next to or on top of one another.

Furthermore, the medical devices 02 are also connected to one another via an additional communication interface 10, which, as the bus system 11 in the example in FIG. 1, is preferably based upon a standard protocol such as the Ethernet protocol.

FIG. 1 already shows that, when the medical devices 02 are arranged as shown, a connection or connection possibility of the adjacent near-field wireless communication interfaces 05 is achieved or becomes possible. According to the disclosure, the detection of this relative arrangement, in particular this direct arrangement of the medical devices 02 one above the other, by means of the near-field wireless communication interfaces 05 leads to the medical devices 02 being transferred to the second operating mode, specifically the coupled operating mode, in which a combined function of the medical devices, viz., a function as a suction-flushing pump, is then achieved. It can be provided that in each case the medical device that detects a connection or connection possibility by means of its near-field wireless communication interface arranged on the base perform the control function or perform the master function, whereas the lower medical device that establishes a connection or detects a connection possibility by means of its upper or cover-side near-field wireless communication interface 05 is also controlled by the upper medical device, for which purpose it sends operating data to the upper medical device and receives control data from the upper medical device 02.

This achieves dynamic role assignment with regard to the control function, in which the upper device or the device that accordingly establishes a connection by means of its base-side near-field wireless communication interface 05 or detects a connection possibility takes over or performs the control function.

The exchanged control and/or operating data can be exchanged both via the near-field wireless communication interfaces 05 and via the additional communication interfaces 10. Real-time data or data for which the shortest possible and fastest possible transmission and provision is important or critical are particularly preferably exchanged via the near-field wireless communication interfaces 05, and further control and/or operating data are exchanged via the additional communication interfaces 10.

Particularly preferably, the additional communication interface 10, as well as the consumables 03 designed as coded hose cassettes, can be used to further increase safety when changing to, and with regard to operation of, the second operating mode.

For example, it can be provided that switching to the second operating mode or operation in the second operating mode only be possible if the communication interfaces 10 and/or the consumables 03 confirm a corresponding operation or verify the possibility of a corresponding operation.

Furthermore, it can be provided that an output via the output units 04 output, in particular display, the operation in the second operating mode.

As soon as the medical devices 02 are arranged relative to one another such that the ranges 09 no longer coincide, the second operating mode is terminated, and each device 02 switches—advantageously, automatically or semi-automatically—to the first operating mode.

FIG. 2 shows an exemplary flowchart for the operation of a medical device 02 according to the present disclosure. In a method step S1, the medical device is put into operation. In a subsequent verification step D1, a check is carried out as to whether another medical device 02 has been identified in a specific relative arrangement, by reading or querying the near-field wireless communication interfaces 05.

If no specific relative arrangement is identified, in method step S2, the medical device 02 is operated in the first operating mode and thus in the individual operating mode. It can optionally be provided that a verification according to method step D1 also take place or be carried out repeatedly in the individual operating mode of the device 02 according to method step S2.

If, in method step D1, the identification of a specific relative arrangement of another medical device is determined, which indicates a need to switch to the second operating mode or initiates, enables, or at least contributes to the process of switching to the second operating mode, in a further method step D2, for example, one or more further checks can be made which aim to verify the transfer to the second operating mode. The verifications in method step D2 can include, for example, a verification via an additional communication interface 10, a user input that may be made upon request, and/or a verification based upon attachments and/or consumables of the medical device 02.

In the event that these additional verification steps or the at least one additional verification step in method step D2 is or are not successfully completed, it can be provided, for example, that the method return to the verification process in method step D1. Alternatively, it can also be provided, as also indicated by dashed lines in the illustration in FIG. 2, that, if the additional verification fails, operation of the medical device in the first operating mode as per method step S2 take place or be initiated.

If the additional verification steps are successfully completed, a further method step D3 can be used to check or decide, for example, whether the medical device in question is to hold or take on the control function, or whether the medical device is to instead also be controlled by another medical device. In this case, a dynamic allocation can preferably be carried out which, as already outlined with reference to FIG. 1, can depend, for example, upon the relative arrangement or positioning of the medical devices 02. In this method step, for example, the second operating mode can also be determined by evaluating the location, type, and/or number of interconnected or interconnectable near-field wireless communication interfaces.

Accordingly, in method steps S3′ and S3″, the medical device 02 can be operated in the second operating mode by not only performing or providing the combined function, but also exchanging corresponding data with another medical device in order to exchange control data and/or operating data. The two variants S3′ and S3″ can represent different second operating modes as well as operation as master or slave.

Furthermore, it can be provided that, even when the medical device is operated in the second operating mode, the method repeatedly or periodically return to step D1 and at least the decision in method step D1, preferably also the decision in method step D2, be checked or verified once again, wherein the second operating mode is preferably terminated, or termination of the second operating mode is at least announced or suggested, if, when method steps D2 and D3 are repeated, they do not indicate or justify the operation of the medical device in the second operating mode.

1. A method for controlling a system comprising at least two medical devices, wherein the at least two medical devices each have at least one near-field wireless communication interface which is arranged in or on the medical devices such that, in at least one specific arrangement of the medical devices relative to one another, a near-field wireless communication interface of one of the at least two medical devices is arranged within the communication range of a near-field wireless communication interface of another of the at least two medical devices, and vice versa, said method having the following method steps:
operating the at least two medical devices in a first independent operating mode in the form of an individual operating mode, in which each of the at least two medical devices carries out or provides an individual function as long as the near-field wireless communication interface does not detect a connection or connection possibility to a further near-field wireless communication interface;
identifying the at least two medical devices in the specific relative arrangement to one another by means of the near-field wireless communication interfaces;
operating the at least two medical devices in a second operating mode in the form of a coupled operating mode, wherein the coupled operating mode of the at least two medical devices performs or provides a combined function that cannot be performed or provided by the at least two medical devices individually when operated in the coupled operating mode if and/or as long as at least one near-field wireless communication interface has established a connection to a further near-field wireless communication interface or detects a connection possibility to a further near-field wireless communication interface.

2. The method according to claim 1, characterized in that the at least two medical devices in a certain relative arrangement are identified as a result of periodically repeated connection attempts, in which at least some of the near-field wireless communication interfaces make an attempt to establish a connection to a further near-field wireless communication interface.

3. The method according to claim 1, characterized in that the second operating mode is determined by the number and/or position and/or type of the near-field wireless communication interfaces which have established a connection to a further near-field wireless communication interface or which detect a connection possibility to a further near-field wireless communication interface.

4. The method according to claim 1, characterized in that, in the second operating mode, a first device of the at least two medical devices performs a control function for at least another one of the at least two medical devices and sends control data to the other one of the at least two medical devices and/or receives operating data from the other one of the at least two medical devices for this purpose.

5. The method according to claim 4, characterized in that the control function is assigned dynamically on the basis of the location or position of the near-field wireless communication interface which has established a connection to a further near-field wireless communication interface or detects a connection possibility to a further wireless communication interface.

6. The method according to claim 4, characterized in that the control function is assigned dynamically on the basis of the type of near-field wireless communication interface which has established a connection to a further near-field wireless communication interface or detects a connection possibility to a further near-field wireless communication interface.

7. The method according to claim 4, characterized in that the control data and/or the operating data are exchanged via the near-field wireless communication interfaces.

8. The method according to claim 7, characterized in that those control data and/or those operating data which, if possible, must be available as real-time data are selectively exchanged via the near-field wireless communication interfaces.

9. The method according to claim 4, characterized in that, when operating at least three medical devices (02) in a second operating mode, at least one of the at least three medical devices comprises two wireless communication interfaces and brings about the transfer of control and/or operating data between the other two of the at least three medical devices.

10. The method according to claim 1, characterized in that control data and/or operating data are exchanged via an additional, preferably wired, communication interface, preferably a BUS system.

11. The method according to claim 1, characterized in that, when changing to or activating the second operating mode, the connection or connection possibility of the near-field wireless communication interfaces is verified via an additional, preferably wired, communication interface.

12. The method according to claim 1, characterized in that, when changing to or activating the second operating mode, verification is carried out by detecting attachments or consumables, specific to the coupled operating mode, of the devices.

13. The method according to claim 1, characterized in that, when changing to or activating the second operating mode, a user input must be made on at least one of the at least two medical devices, preferably a device of the at least two medical devices that performs or fulfills the control function in the second operating mode, to confirm the change in operating mode, and the at least one of the at least two medical devices preferably issues a request for a user input via an output unit for this purpose.

14. The method according to claim 1, characterized in that, when changing to or activating the second operating mode, an output, preferably a display, on an output unit of at least one device of the at least two medical devices is made, which outputs or reveals to a user that operation is taking place in the second operating mode.

15. The method according to claim 1, characterized in that, depending upon the operating mode of a medical device of the at least two medical devices, an output unit and/or an input unit, preferably a combined input and output unit, is adapted to the operating mode in order to enable settings and operating data to be output and/or input in a manner adapted to the corresponding operating mode.

16. The method according to claim 1, characterized in that a first device and a second device of the at least two medical devices comprise a pump, preferably a roller pump, and a pump functionality is provided in the first operating mode, wherein a suction-flushing-pump functionality is provided in the second operating mode.

17. The method according to claim 1, characterized in that a third medical device comprises a hose valve and provides a valve functionality in the first and second operating modes.

18. A medical system comprising at least two medical devices, each having at least one near-field wireless communication interface, a control apparatus, and a data memory, and which are configured to execute computer programs to carry out a method according to claim 1.
